(19) United States
(12) Patent Application Publication
(10) Pub. No.: US 2022/0354412 A1
(43) Pub. Date: Nov. 10, 2022

(54) MYOPOTENTIAL MEASUREMENT APPARATUS AND MYOPOTENTIAL MEASUREMENT WEARABLE EQUIPMENT

(71) Applicant: SHINKO ELECTRIC INDUSTRIES CO., LTD., Nagano (JP)

(72) Inventor: Hisashi Kaneda, Nagano (JP)

(21) Appl. No.: 17/728,508

(22) Filed: Apr. 25, 2022

(30) Foreign Application Priority Data
May 6, 2021 (JP) 2021-078705

(51) Int. Cl. A61B 5/389; A61B 5/00

(52) U.S. Cl. CPC A61B 5/389; A61B 5/6804

(57) ABSTRACT

A myopotential measurement apparatus includes a wearable unit that is wearable on a human body, a pair of electrodes that is formed by embroidering the wearable unit with conductive threads, where the electrodes face each other and have adjacent one ends that protrude highest from the wearable unit, and a measurement unit that measures potentials that are generated by a measurement target that comes into contact with the pair of electrodes.

### CROSS-REFERENCE TO RELATED APPLICATION

This application is based upon and claims the benefit of priority of the prior Japanese Patent Application No. 2021-078705, filed on May 6, 2021, the entire contents of which are incorporated herein by reference.

### FIELD

The embodiment discussed herein is related to a myopotential measurement apparatus and a myopotential measurement wearable equipment.

### BACKGROUND

Conventionally, in the medical practice, such as rehabilitation, an apparatus that measures myopotentials that are generated with motion of muscles may be used in some cases. The apparatus as described above measures action potentials that are generated by action of muscle fibers, by bringing two or three electrodes into contact with skin in the vicinity of a measurement target muscle.

To stably measure myopotentials, it is desirable to fix the electrodes while bringing the electrodes into close contact with skin, and therefore, for example, wet electrodes that include adhesive layers may be used as electrodes in a medical myopotential measurement apparatus. However, if the wet electrodes are brought into close contact with skin for a long time, a problem, such as skin rash, may occur; therefore, there is a need for a dry electrode in which the adhesive layer is not used.

As the dry electrode, for example, a textile electrode that is formed in a part of clothes by using a conductive thread has been studied.

Patent Literature 1: Japanese Translation of PCT International Application Publication No. 2020-512095

Patent Literature 2: Japanese Laid-open Patent Publication No. 2018-061778

However, when myopotentials are measured by using the dry electrode, there is a problem in that it is difficult to perform measurement stably. Specifically, if the textile electrode that is formed in clothes is used for example, the textile electrode may be separated from skin along with motion of a human body, and it may become impossible to measure myopotentials.

Further, it may be possible to relatively increase an area of the textile electrode to prevent the entire textile electrode from being separated from skin; however, in this case, a contact position at which the textile electrode comes into contact with skin may vary. As a result, a distance between contact positions of a plurality of electrodes that measure potentials may vary, so that it becomes difficult to accurately measure myopotentials.

### SUMMARY

According to an aspect of an embodiment, a myopotential measurement apparatus includes: a wearable unit that is wearable on a human body; a pair of electrodes that is formed by embroidering the wearable unit with conductive threads, the electrodes facing each other and having adjacent one ends that protrude highest from the wearable unit; and a measurement unit that measures potentials that are generated by a measurement target that comes into contact with the pair of electrodes.

The object and advantages of the invention will be realized and attained by means of the elements and combinations particularly pointed out in the claims.

It is to be understood that both the foregoing general description and the following detailed description are exemplary and explanatory and are not restrictive of the invention, as claimed.

### BRIEF DESCRIPTION OF DRAWINGS

FIG. 1 is a diagram illustrating an external appearance of a myopotential measurement apparatus according to one embodiment;

FIG. 2 is a diagram illustrating a configuration of the myopotential measurement apparatus according to one embodiment;

FIG. 3 is a plan view illustrating a configuration of a wearable unit;

FIGS. 4A and 4B are diagrams illustrating a specific example of a myopotential measurement state;

FIG. 5 is a diagram illustrating a modification of the myopotential measurement apparatus;

FIG. 6 is a diagram illustrating another modification of the myopotential measurement apparatus; and

FIG. 7 is a diagram illustrating still another modification of the myopotential measurement apparatus.

### DESCRIPTION OF EMBODIMENTS

One embodiment of a myopotential measurement apparatus and a myopotential measurement wearable equipment disclosed in the present application will be described in detail below with reference to the drawings. The present invention is not limited by the embodiment below.

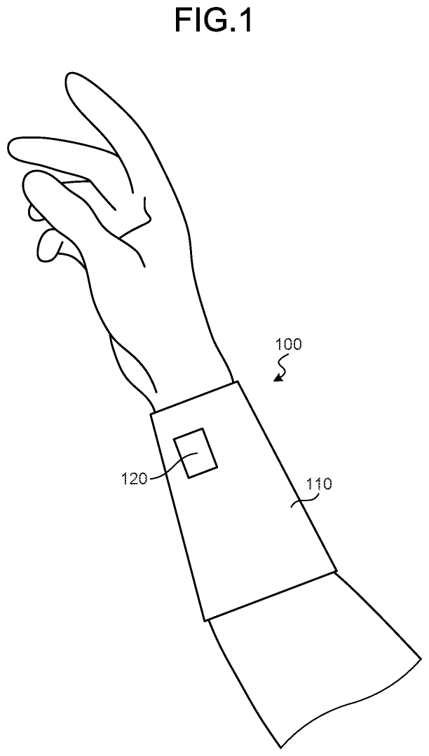

FIG. 1 is a diagram illustrating an external appearance of a myopotential measurement apparatus 100 according to one embodiment. Here, the external appearance of the myopotential measurement apparatus 100 that measures myopotentials of a muscle of an arm is illustrated. While a case in which myopotentials of a muscle of an arm are measured will be described in the present embodiment, the myopotential measurement apparatus according to the present invention is able to measure myopotentials of muscles of other parts different from the arm.

The myopotential measurement apparatus 100 illustrated in FIG. 1 includes a wearable unit 110 and a measurement unit 120.

The wearable unit 110 is a myopotential measurement wearable equipment that is made with a base material that is wearable on a human body, and that is worn in the vicinity of a muscle for which myopotentials are to be measured. Specifically, the wearable unit 110 is formed by forming a stretchable cloth containing chemical fibers, such as polyurethane, polyester, or polyamide, into a certain shape corresponding to a part of a human body on which the wearable unit is to be worn. In this example, the myopotential measurement apparatus 100 is worn on the arm, so that the wearable unit 110 is formed by forming a stretchable cloth into a cylindrical shape. Further, as will be described later, a pair of electrodes is formed using conductive threads in a portion in which the measurement unit 120 is mounted on the wearable unit 110.

The measurement unit 120 is mounted at a position at which the pair of electrodes is formed on the wearable unit 110, and measures potentials between the electrodes on the basis of an electric current that flows through the electrodes that come into contact with the human body. In other words, the measurement unit 120 measures myopotentials of a measurement target muscle that is located at the position at which the pair of electrodes is formed. The measurement unit 120 is formed by, for example, mounting an integrated circuit (IC) chip on a circuit substrate.

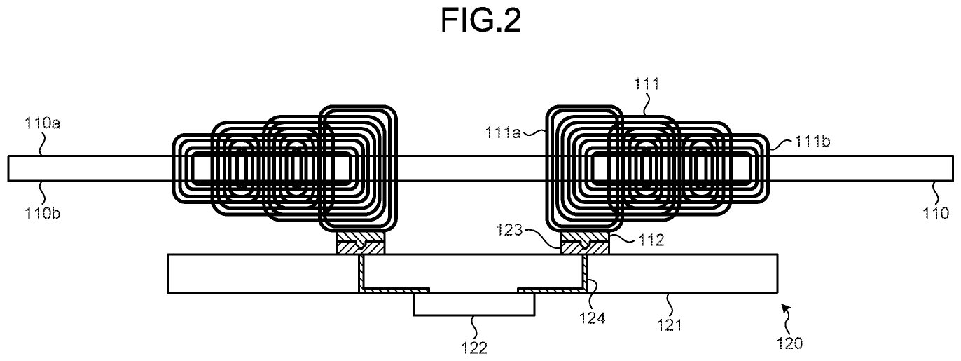

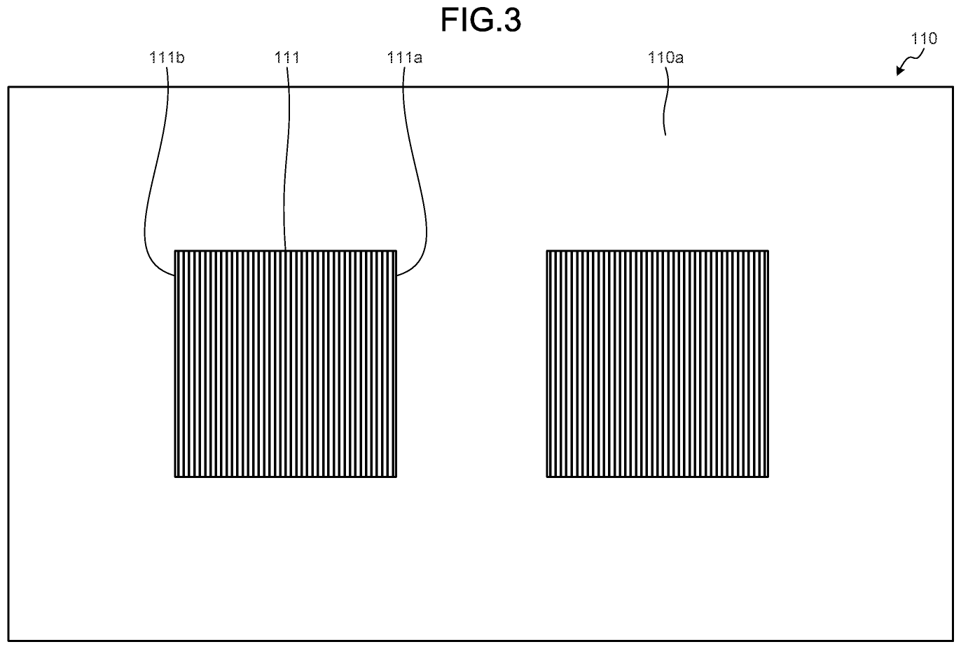

FIG. 2 is a diagram illustrating a configuration of the myopotential measurement apparatus 100 according to one embodiment. Specifically, FIG. 2 schematically illustrates a cross section of the myopotential measurement apparatus 100 at the position of the measurement unit 120. FIG. 3 is a plan view illustrating a configuration of a wearable unit.

As illustrated in FIGS. 2 and 3, electrodes 111 that face each other are formed, as a pair, on the wearable unit 110. The electrodes 111 as a pair face each other with an interval of about 10 millimeters (mm), for example. The electrodes 111 are formed by embroidering the base material of the wearable unit 110 with conductive threads. Therefore, the electrodes 111 protrude from a surface 110a (hereinafter, referred to as an “inner surface”) of the base material of the wearable unit 110 that comes into contact with the human body and another surface 110b (hereinafter, referred to as an “outer surface”) on which the measurement unit 120 is mounted. Further, the measurement unit 120 is connected to the electrodes 111 at a side of the outer surface 110b.

The electrodes 111 are dry electrodes that are formed by embroidering loops across the inner surface 110a side and the outer surface 110b side of the base material of the wearable unit 110 with conductive threads. At this time, the number of loops made with the conductive threads is increased with approach to adjacent one ends 111a of the electrodes 111 as a pair. With this configuration, the electrodes 111 are shaped such that heights from the inner surface 110a and the outer surface 110b increase from distant one ends 1l1b of the electrodes 111 as a pair to the adjacent one ends 111a. In other words, the electrodes 111 protrude highest from the inner surface 110a and the outer surface 110b of the base material at the adjacent one ends 11a. For example, the electrodes 111 protrude from the inner surface 110a of the base material by about 4 mm at the one ends 111a. In contrast, the electrodes 111 protrude from the inner surface 110a of the base material by about 1 mm at the one ends 111b.

In this manner, the electrodes 111 protrude highest at the adjacent one ends 11a, so that when the myopotential measurement apparatus 100 is worn on a human body, the one ends 111a of the electrodes 111 as a pair continuously come into contact with the human body at the side of the inner surface 110a of the base material. As a result, it is possible to stably measure myopotentials without causing a change to occur in a distance between the electrodes that come into contact with the human body. Further, surfaces of the electrodes 111 that come into contact with the human body are inclined, so that when wearing the myopotential measurement apparatus 100 on the human body, it is possible to easily wear the myopotential measurement apparatus 100.

Convex buttons 112 are fixed to the one ends 111a of the electrodes 111 at the side of the outer surface 110b. The convex buttons 112 are made with a conductive body, such as stainless steel (SUS), and by engaging with concave buttons 123 that are fixed to the measurement unit 120, the convex buttons 112 fix the measurement unit 120 to the wearable unit 110 and electrically connect the electrodes 111 and the measurement unit 120.

The measurement unit 120 is mounted on the outer surface 110b side of the base material of the wearable unit 110 at the position at which the electrodes 111 are formed. The measurement unit 120 includes a circuit substrate 121, a processing circuit 122, the concave buttons 123, and wires 124.

The circuit substrate 121 is, for example, a flexible substrate with flexibility, and is a substrate that is deformable along with the base material of the wearable unit 110. The processing circuit 122 is mounted on a surface of the circuit substrate 121 at a side opposite to the wearable unit 110. The processing circuit 122 measures potentials between the electrodes 111 on the basis of an electric current that flows through the electrodes 111 as a pair. In other words, the processing circuit 122 measures myopotentials of parts that come into contact with the electrodes 111.

The concave buttons 123 as a pair are fixed to a surface of the circuit substrate 121 at the side of the wearable unit 110. The concave buttons 123 are arranged at certain positions corresponding to the convex buttons 112 that are fixed to the electrodes 111 as a pair. Further, the measurement unit 120 is mounted on the wearable unit 110 by engaging the convex buttons 112 fixed to the electrodes 111 with the corresponding concave buttons 123. The concave buttons 123 are made with a conductive body, such as stainless steel (SUS) similarly to the convex buttons 112, and by engaging with the convex buttons 112, the concave buttons 123 are electrically connected to the electrodes 111.

The processing circuit 122 and the concave buttons 123 are connected by the wires 124 that penetrate through the circuit substrate 121. The wires 124 are made with copper, copper alloy, or the like, and electrically connect the concave buttons 123 and terminals of the processing circuit 122. With this configuration, the processing circuit 122 is electrically connected to the pair of electrodes 111 and able to measure potentials between the electrodes 111.

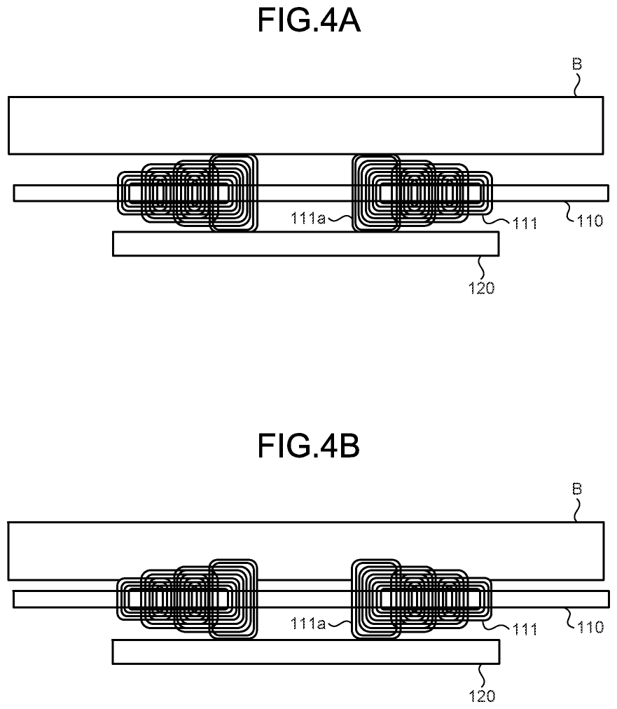

FIGS. 4A and 4B are diagrams schematically illustrating a state in which the myopotential measurement apparatus 100 is worn on a human body and measures myopotentials.

As illustrated in FIG. 4A, if the myopotential measurement apparatus 100 is worn on a human body B, the electrodes 111 come into contact with the human body B at the adjacent one ends 11a of the electrodes 111 because the one ends 11a protrude high from the base material of the wearable unit 110. The one ends 111a continuously come into contact with the human body B even if other portions of the electrodes 111 do not come into contact with the human body B, so that it is possible to maintain a constant interval between contact positions of the electrodes 111, as a pair, at which the electrodes 111 come into contact with the human body B, and the measurement unit 120 is able to measure myopotentials of parts that come into contact with the electrodes 111 in a state in which the distance between the electrodes remains unchanged.

Further, as illustrated in FIG. 4B, even if the wearable unit 110 approaches the human body B and the entire electrodes 111 come into contact with the human body B, the distance between the electrodes remains unchanged as the distance between the one ends 111a of the electrodes 111 that face each other, so that it is possible to measure myopotentials under the same condition as in the state illustrated in FIG. 4A. In other words, it is possible to stably measure myopotentials by using the dry electrodes. Furthermore, when the entire electrodes 111 come into contact with the human body B, it is possible to reduce noise that is generated in the electrodes 111, so that it is possible to more accurately measure myopotentials.

As described above, according to the present embodiment, when the pair of electrodes for measuring myopotentials is formed by embroidering a stretchable base material with conductive threads, the electrodes are formed so as to protrude highest from a surface of the base material at the adjacent one ends. Therefore, it is possible to continuously bring the most protruding one end of each of the electrodes into contact with a human body, and maintain a constant inter-electrode distance that is an interval between positions at which the electrodes as a pair come into contact with the human body. As a result, it is possible to stably measure myopotentials by using the dry electrodes.

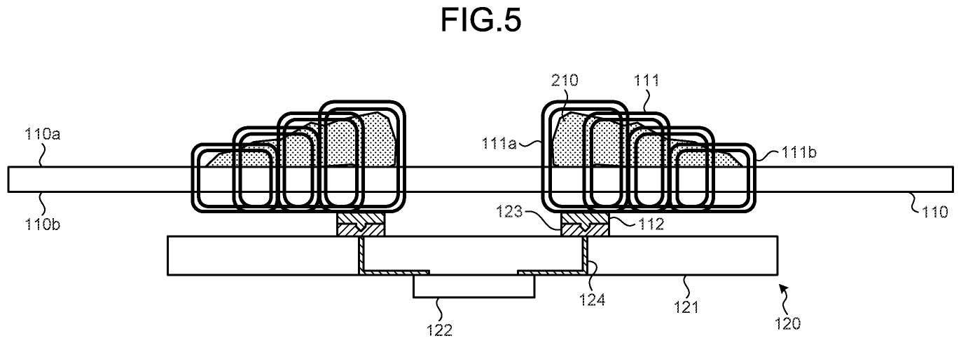

While the one ends 111a of the electrodes 111 are caused to protrude highest by increasing the number of loops made with the conductive threads in one embodiment as described above, it may be possible to arrange a support member on the inner surface 110a of the base material of the wearable unit 110 and cause the electrodes 111 to protrude by embroidering the base material and the support member with the conductive threads. Specifically, as illustrated in FIG. 5 for example, a pair of support members 210 that is made of a soft material, such as cotton, is arranged on the inner surface 110a of the base material of the wearable unit 110. The support members 210 as a pair face each other and are shaped such that heights from the inner surface 110a are highest at adjacent one ends of the support members 210.

Further, the pair of electrodes 111 is formed by embroidering loops across surfaces of the support members 210 and the outer surface 110b of the base material of the wearable unit 110 with conductive threads. At this time, the number of loops made with the conductive threads is constant at any position of the electrodes 111. The electrodes 111 are shaped such that heights from the inner surface 110a increases form the distant one ends 111b to the adjacent one ends 11a of the electrodes 111 as a pair, in accordance with the shapes of the support members 210. In other words, the electrodes 111 protrude highest from the inner surface 110a of the base material at the adjacent one ends 111a.

With this configuration, even with embroidery of the same number of loops with the conductive threads, it is possible to implement the myopotential measurement apparatus 100 in which the adjacent one ends 111a of the electrodes 111 as a pair continuously come into contact with a human body. Further, according to the myopotential measurement apparatus 100, a distance between the electrodes that come into contact with the human body remains unchanged, so that it is possible to stably measure myopotentials.

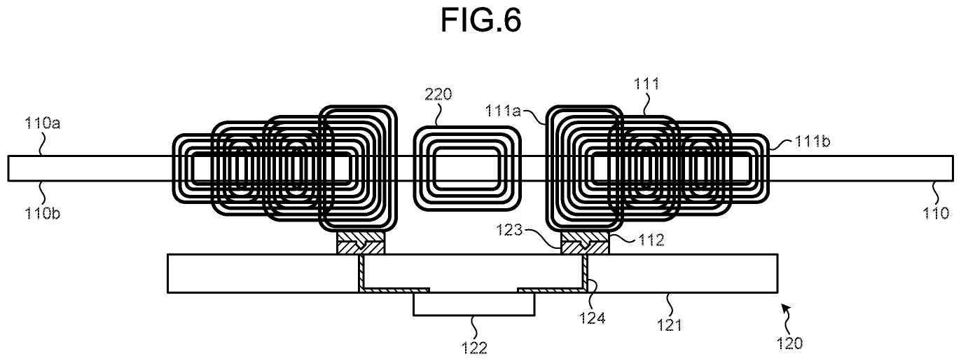

Furthermore, in one embodiment as described above, it may be possible to embroider between the electrodes 111 as a pair with non-conductive normal threads having low stretching property in order to reinforce the base material of the wearable unit 110. Specifically, as illustrated in FIG. 6 for example, it may be possible to form a reinforcing portion 220 in a region sandwiched between the pair of electrodes 111 on the wearable unit 110. The reinforcing portion 220 is formed by embroidering loops across the inner surface 110a side and the outer surface 110b side of the base material of the wearable unit 110 with threads made of normal fibers. The reinforcing portion 220 protrudes from the wearable unit 110 within a range that does not exceed the heights of the one ends 111a of the electrodes 111 as a pair.

As described above, with formation of the reinforcing portion 220 between the electrodes 111, the base material between the electrodes 111 as a pair is reinforced and less likely to be deformed, so that it is possible to reliably maintain a constant distance between the electrodes. Furthermore, by arranging the reinforcing portion 220 between the pair of electrodes 111 such that the reinforcing portion 220 comes into contact with the electrodes 111 and connects the electrodes 111, it is possible to prevent a change in the distance between the electrodes even if the wearable unit 110 is stretched. As a result, the distance between the electrodes that come into contact with the human body remains unchanged, so that it is possible to stably measure myopotentials.

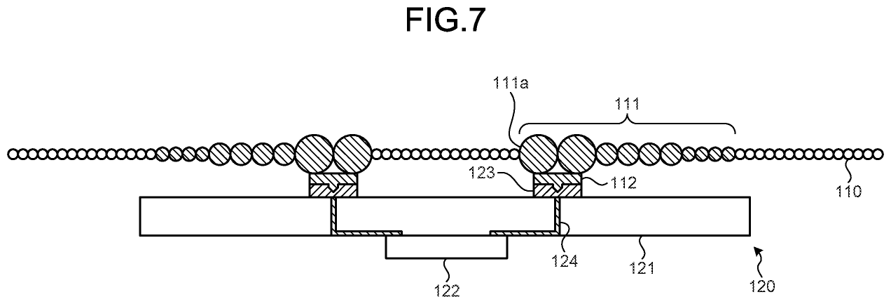

Moreover, while the wearable unit 110 is formed by embroidering the base material with the conductive threads in one embodiment as described above, the entire wearable unit 110 may be formed by weaving threads. In this case, as illustrated in FIG. 7 for example, a portion of the wearable unit 110 other than the pair of electrodes 111 is formed by weaving threads made of normal fibers, and a portion corresponding to the electrodes 111 is formed by weaving conductive threads. Furthermore, as for the pair of electrodes 111, the adjacent one ends 111a are caused to protrude highest by using conductive threads with different thicknesses. Specifically, the electrodes 111 are formed by weaving thicker conductive threads with approach to the adjacent one ends 111a of the pair of electrodes 111.

As described above, it is possible to form the pair of electrodes 111 in which the adjacent one ends 111a protrude highest by weaving conductive threads with different thicknesses, instead of embroidery with conductive threads. Further, for example, it may be possible to form the pair of electrodes 111 in which the adjacent one ends 111a protrude highest by depositing or printing the pair of electrodes 111, which is formed as a separate body by using the conductive threads, onto the inner surface 110a of the base material of the wearable unit 110. In this case, it may be possible to sew, with the conductive threads, the convex buttons 112 on the outer surface 110b of the base material at positions corresponding to the electrodes 111 to be deposited or printed, and allow the electrodes 111 at the side of the inner surface 110a and the convex buttons 112 at the side of the outer surface 110b to be electrically connected to each other.

Furthermore, while the pair of electrodes 111 in which the adjacent one ends 111a protrude highest is formed in one embodiment as described above, it may be possible to form a ground electrode in addition to the pair of electrodes 111. The ground electrode may be formed by embroidering with conductive threads, similarly to the pair of electrodes 111. Moreover, the ground electrode may have a constant height from the inner surface 110a of the base material of the wearable unit 110 within a range that does not exceed the heights of the one ends 111a of the electrodes 111 as a pair.

According to one embodiment of the myopotential measurement apparatus and the myopotential measurement wearable equipment disclosed in the present application, it is possible to stably measure myopotentials by using dry electrodes.

All examples and conditional language recited herein are intended for pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although the embodiments of the present invention have been described in detail, it should be understood that the various changes, substitutions, and alterations could be made hereto without departing from the spirit and scope of the invention.

What is claimed is:

1. A myopotential measurement apparatus comprising:
a wearable unit that is wearable on a human body;
a pair of electrodes that is formed by embroidering the wearable unit with conductive threads, the electrodes facing each other and having adjacent one ends that protrude highest from the wearable unit; and
a measurement unit that measures potentials that are generated by a measurement target that comes into contact with the pair of electrodes.

2. The myopotential measurement apparatus according to claim 1, wherein the pair of electrodes is formed by embroidering loops with conductive threads such that number of the loops increases with approach to the adjacent one ends from distant one ends of the electrodes.

3. The myopotential measurement apparatus according to claim 1, wherein the pair of electrodes is formed by embroidering loops across a support member and the wearable unit with conductive threads, the support member being arranged on the wearable unit at a side that comes into contact with a human body and protruding highest from the wearable unit at the adjacent one ends.

4. The myopotential measurement apparatus according to claim 1, further comprising:
a reinforcing portion that is formed by embroidering a region that is sandwiched between the pair of electrodes on the wearable unit with non-conductive threads.

5. The myopotential measurement apparatus according to claim 4, wherein the reinforcing portion protrudes from the wearable unit within a range that does not exceed most protruding one ends of the pair of electrodes.

6. The myopotential measurement apparatus according to claim 1, further comprising:
first buttons that are conductive and that are fixed to the pair of electrodes at a side opposite to a side that comes into contact with the measurement target; and
second buttons that are fixed to the measurement unit and that are engageable with the first buttons.

7. A myopotential measurement wearable equipment comprising:
a wearable unit that is wearable on a human body; and
a pair of electrodes that is formed by embroidering the wearable unit with conductive threads, the electrodes facing each other and having adjacent one ends that protrude highest from the wearable unit.

8. The myopotential measurement wearable equipment according to claim 7, wherein the pair of electrodes is formed by embroidering loops with conductive threads such that number of the loops increases with approach to the adjacent one ends from distant one ends of the electrodes.

9. The myopotential measurement wearable equipment according to claim 7, wherein the pair of electrodes is formed by embroidering loops across a support member and the wearable unit with conductive threads, the support member being arranged on the wearable unit at a side that comes into contact with a human body and protruding highest from the wearable unit at the adjacent one ends.

10. The myopotential measurement wearable equipment according to claim 7, further comprising:
a reinforcing portion that is formed by embroidering a region that is sandwiched between the pair of electrodes on the wearable unit with non-conductive threads.
